(19) United States
(12) Patent Application Publication
(10) Pub. No.: US 2008/0140182 A1
(43) Pub. Date: Jun. 12, 2008

(54) Composite endoluminal prostheses for treating vulnerable plaque

(76) Inventor: Patricia Scheller, Solebury, PA (US)

Correspondence Address: PATTON BOGGS LLP, 8484 WESTPARK DRIVE, SUITE 900, MCLEAN, VA, 22102, US

(21) Appl. No.: 11/790,124

(22) Filed: Apr. 24, 2007

Related U.S. Application Data
(60) Provisional application No. 60/795,576, filed on Apr. 28, 2006.

(51) Int. Cl. A61F 2/06

(52) U.S. Cl. 623/1.17; 623/1.15; 623/1.16

(57) ABSTRACT

The invention provides expandable tubular endoluminal prostheses for the treatment of atherosclerotic lesions, such as vulnerable plaques, and methods for treating such lesions using the prostheses. The endoprostheses may include at least two expandable ring-like elements disposed on the inside or about the outer surface of an at least substantially tubular biodegradable element. The ring-like elements may be radio-opaque and may have a sinuate form. In use, a prosthesis according to the invention is expanded in a blood vessel so that the tubular biodegradable element at least partially covers an atherosclerotic lesion, such as a vulnerable plaque.

This application claims the benefit of U.S. provisional patent application Ser. No. 60/795,576 filed Apr. 28, 2006, which is incorporated herein by reference in its entirety.

### FIELD OF THE INVENTION

The invention relates generally to the fields of expandable endoluminal vascular prostheses and their use in treating atherosclerotic lesions. BACKGROUND OF INVENTION

Vulnerable plaques, which are sometimes referred to as high-risk atherosclerotic plaques, are arterial atherosclerotic lesions characterized by a subluminal thrombotic lipid-rich pool of materials contained by a thin fibrous cap. Although vulnerable plaques are non-stenotic or nominally stenotic, it is believed that their rupture, resulting in the release of thrombotic contents, accounts for a significant fraction of adverse cardiac events.

U.S. Publication No. 2002/0004679 discloses drug eluting polymer stents for treating restenosis with topoisomerase inhibitors, and is incorporated herein by reference in its entirety.

U.S. Publication No. 2003/0009213 discloses stents having a drug-eluting cover for the treatment of vulnerable plaque and manners of affixing the cover to the stent, and is incorporated herein by reference in its entirety.

U.S. Publication No. 2003/0125799 discloses intravascular stents for the treatment of vulnerable plaque that consist of opposing end ring portions and a central strut portion having a zig-zag configuration that connects with the end portion at apices of the zig-zag structure, and is incorporated herein by reference in its entirety. The particular zig-zag structure of the stent tends to cause substantial foreshortening upon radial expansion of the device.

U.S. Publication No. 2005/0038503 discloses various filament-based endovascular prostheses, and is hereby incorporated by reference herein in its entirety.

U.S. Publication No. 2005/0137678 discloses a low-profile resorbable polymer stent and compositions therefore, and is incorporated herein by reference in its entirety.

U.S. Publication No. 2005/0228473 discloses frame-like devices for delivering treatments to sites within arteries, and is hereby incorporated by reference herein in its entirety.

U.S. Publication No. 2005/0287184 discloses drug-delivery stent formulations for treating restenosis and vulnerable plaque, and is hereby incorporated by reference herein in its entirety.

### SUMMARY OF INVENTION

The present invention provides tubular endoluminal prostheses and methods of use thereof for treating atherosclerotic lesions such as vulnerable plaques.

One embodiment of the invention provides an expandable endovascular prosthesis for the treatment of vulnerable plaques that includes at least two expandable annular elements (ring-like), each having a central axis; and an at least substantially tubular biodegradable element having an inner surface, an outer surface and a longitudinal central axis. The central axis of each of the sinuate annular elements and of the tubular biodegradable element are aligned so that the composite device has a tubular profile. At least one of the expandable annular elements may be at least substantially sinuate in form. At least some of the expandable annular elements may be at least partially radiopaque to facilitate proper placement of the prosthesis within a blood vessel.

A further embodiment of the invention provides a method for treating vulnerable plaque in a patient in need thereof that includes the step of: deploying an endoprosthesis according to the invention at a site of a vulnerable plaque in a blood vessel of a patient. The biodegradable element of the prosthesis may be drug-eluting or not drug-eluting.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 illustrates an embodiment of a prosthesis according to the invention which has four sinuate annular segments covered by a biodegradable tubular element.

FIG. 2 shows a “rolled out” view of the prosthesis shown in FIG. 1.

### DETAILED DESCRIPTION

The invention provides tubular endovascular prostheses for the treatment of vulnerable plaque and methods of treatment using the endoprostheses.

One embodiment of the invention provides an expandable endovascular prosthesis for the treatment of vulnerable plaques that includes: at least two expandable annular elements, each having a central axis; and an at least substantially tubular biodegradable element having an inner surface, an outer surface and a longitudinal central axis. The central axis of each of the sinuate annular elements and of the biodegradable tubular element are aligned so that the prosthesis as a whole has an at least substantially tubular form. One or more of the expandable annular elements may be at least substantially tubular or ring-like in form. The at least two expandable annular elements may be fixed or connected to the tubular biodegradable element. In one variation, of the at least two expandable annular elements, one is disposed at or near one end of the tubular biodegradable element and another is disposed at or near the opposite end of the tubular biodegradable element. In another variation, there are only two expandable annular elements, one at or near each end of the tubular biodegradable element. In still another variation, there are three expandable annular elements, with one being located one at or near each of the ends of the tubular biodegradable element

In one variation, the biodegradable tubular element is disposed within the expandable annular elements. In another variation, at least two of the expandable annular elements surround the biodegradable tubular element.

In another variation, the expandable annular elements are disposed within the biodegradable tubular element. In another variation, at least two of the expandable annular elements are disposed within the lumen of the biodegradable tubular element.

One or more of the expandable annular elements may be at least partially radio-opaque. One or more of the expandable annular elements may be at least partially metallic. The biodegradable tubular element may be less radio-opaque than the at least partially expandable annular elements. The biodegradable tubular element may, for example, be at least substantially radio-transparent.

The composite prosthesis formed by the biodegradable tubular element and the expandable annular elements may be at least partially balloon expandable. One or more of the biodegradable tubular element and/or at least one of the expandable annular elements may be self-expanding. In this manner, the composite prosthesis may be at least partially self-expanding.

Various aspects of the invention are described below with reference to the appended figures.

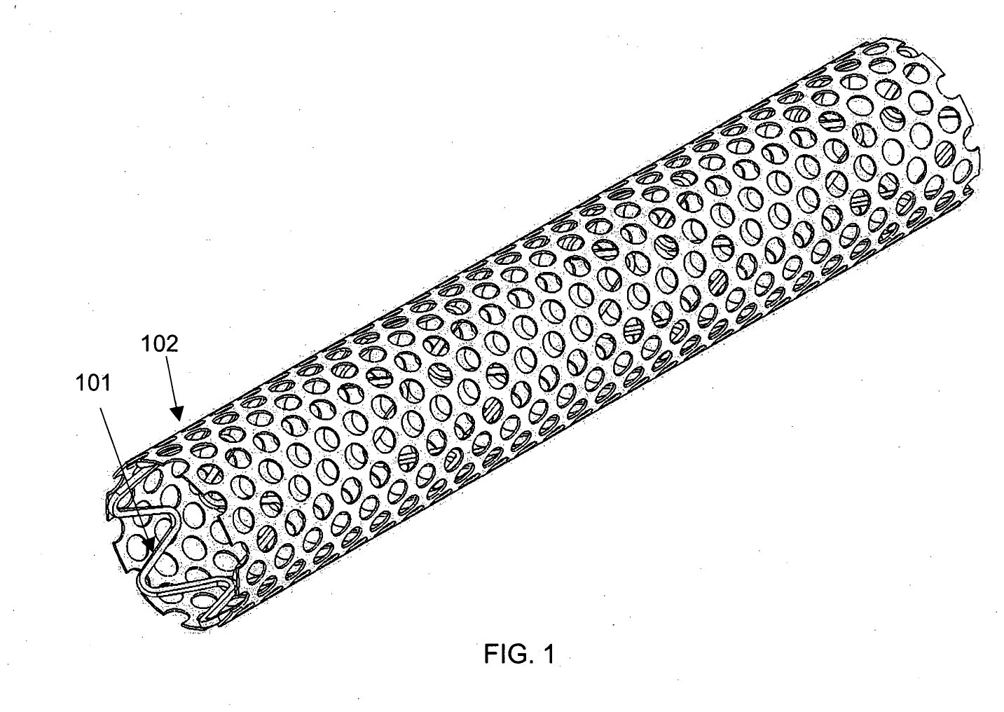

FIG. 1 illustrates an embodiment of a prosthesis according to the invention which has four sinuate expandable annular segments covered by a biodegradable tubular element. Each of the sinuate annular elements, a first being shown as 101, is metallic and radiopaque while the biodegradable tubular element 102 is radio-transparent. In the embodiment shown, each annular element is composed of a single filament and is not connected to neighboring annular elements by struts. Not connecting neighboring annular elements with struts, or minimizing such connections, increases the conformability of the device. The holes in the tubular element may, for example, be 500 microns in diameter, as represented in the figure, or less. The biodegradable tubular element may be polymeric and drug loaded. The edge of the biodegradable tubular element at each end of the prosthesis may be as shown or may, for example, be smooth.

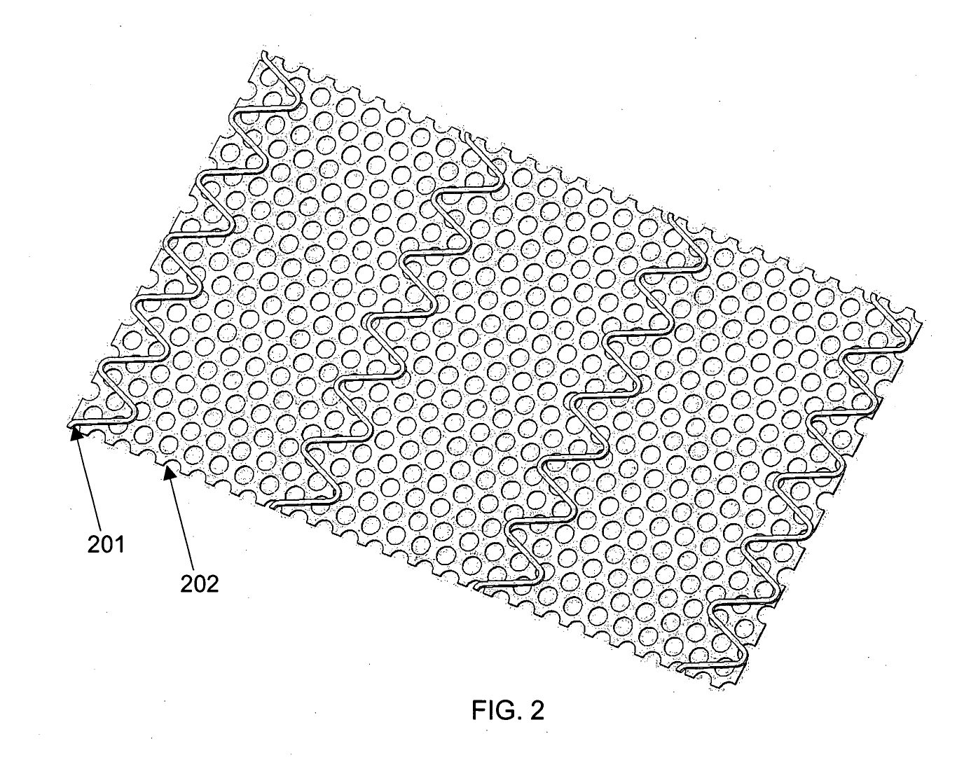

FIG. 2 shows a “rolled out” view of the prosthesis shown in FIG. 1, as if the tubular prosthesis were sliced along its longitudinal axis and flattened. The tubular prosthesis may be, but is not necessarily, formed from flat elements as shown in FIG. 2 which are rolled into the respective annular and tubular configurations of the prosthesis. Element 201 is the first sinuate annular element of four such elements and element 202 is the biodegradable tubular element of the prosthesis.

The expandable annular elements of the prostheses of the invention may be metallic and/or polymeric in composition. Suitable metals include, but are not limited to stainless steel, titanium, titanium alloys, platinum and gold. Shape-memory metal alloys may be used to produce self-expanding versions of endoprostheses according to the invention. For example, suitable shape-memory alloys include, but are not limited, to Nitinol and Elgiloy. The expandable annular elements may be biodegradable or non-biodegradable. For example, at least one or all of the expandable annular elements may be non-biodegradable. In one embodiment, at least one of the expandable annular elements is biodegradable, but the biodegradable annular element(s) biodegrade at a rate substantially slower than the rate at which the biodegradable tubular element biodegrades. This may be achieved, for example, by using a more slowly degrading polymer or polymer blend for the expandable annular elements than for the biodegradable tubular element or by making the expandable annular elements thicker than the biodegradable tubular element such as when both are composed of the same polymer composition.

Any type of biodegradable polymers, biodegradable polymer blends and/or biodegradable metals or metal alloys may be used according to the invention for the biodegradable tubular element, and for the expandable annular elements (in embodiments in which the annular elements are biodegradable). As used herein, the term “biodegradable” should be construed broadly as meaning that the polymer(s) or metal(s) will degrade, erode and/or corrode once placed within a patient's body. Biodegradable metals include, but are not limited to, magnesium and iron and their biodegradable alloys, as known in the art. Biodegradable polymers as referred to herein also include bioerodable and bioresorbable polymers.

Suitable types of biodegradable polymer materials for use in the invention include, but are not limited to, polyester, polyanhydride, polyamide, polyurethane, polyurea, polyether, polysaccharide, polyamine, polyphosphate, polyphosphonate, polysulfonate, polysulfonamide, polyphosphazene, hydrogel, polylactide, polyglycolide, protein cell matrix, or copolymer or polymer blend thereof.

Homopolymers of polylactic acid (PLA), for example PLLA, PDLA and poly(D,L,)lactic acid, stereopolymers thereof, and copolymer of PLA with other polymeric units such as glycolide provide a number of characteristics that are useful in a polymeric endoprosthesis for treating a lesion of a blood vessel such as a high risk atherosclerotic plaque (vulnerable plaque). First, polymers made of these components biodegrade in vivo into harmless compounds. PLA is hydrolyzed into lactic acid in vivo. Second, these polymers are well suited to balloon-mediated expansion using a delivery catheter. Third, polymers made of these materials can be imparted with a shape-memory so that polymeric, at least partially self-expanding, tubular endoprostheses can be provided. Self-expanding polymeric prostheses according to the invention may also, for example, be at least partially balloon-expanded. Methods for producing biodegradable, polymeric shape-memory endoprostheses are described, for example, in U.S. Pat. Nos. 4,950,258, 5,163,952, and 6,281,262, each of which is incorporated by reference herein in its entirety.

Endoprostheses according to the invention may be manufactured by any suitable method. For example, the expandable annular elements may be produced by laser cutting the elements from a tubular metallic blank or a tubular polymeric blank. Methods for forming metallic and polymeric tubular blanks are well known. For example, sputtering metallic material onto a mandrel may be used. In another example, the shape of the expandable annular elements can be laser cut or stamped out of a flat sheet of metallic material and then formed and welded into a ring-like configuration. Once formed into shape, metallic expandable annular elements may optionally be electrochemically polished and/or etched. The expandable annular sections may be formed separately by, for example, laser cutting from a metallic tubular blank or by winding a filament or band of a metallic material about a suitable cylindrical jig. The ends of such a jig-wound expandable annular section may, for example, be welded together to form a continuous ring structure.

The expandable annular elements may be independent of neighboring expandable elements, i.e., not joined to each other by struts or other connective elements, or at least some of the neighboring expandable elements may be joined to one another by one or more struts and/or other connective elements, such as bands. The connective elements referred to herein do not include the tubular biodegradable element. For example, in embodiments in which at least some of the neighboring annular elements are connected by one or more struts, the struts, which may for example be segments of filament, may be formed in the process of forming the annular elements themselves, such as by laser cutting from a tubular blank, or may be joined to the annular elements by welding, melting, physical attachment or interlocking and/or by adhesive binding. In a related embodiment, the one or more struts or other connective elements that may connect neighboring annular elements are sized, configured and of number to provide at least 80% open space, at least 90% open space, or at least 95% open space in a section between connected annular elements as measured by the area taken up by the connective elements in the section when the prosthesis is in its expanded state divided by the area of the wall that would be formed by a solid tube having the same expansion radius and length as the section.

The biodegradable tubular element may, for example, be a continuous porous or non-porous polymeric structure or it may be a braid, woven, or knit polymeric structure. The biodegradable tubular element of a prosthesis according to the invention may optionally be laser cut from a tubular blank, such as one formed by extrusion molding. In this manner, a selected porosity and/or cell pattern can be established in the wall of the tubular element. The biodegradable tubular element may be at least partially self-expanding, for example as the result of a shape-memory characteristic. The biodegradable tubular element may, for example, be thermoplastically expandable but not be self-expanding. The wall of the biodegradable tubular element may be porous or non-porous. The biodegradable tubular element may have its own radial resiliency or its ability to remain in a radially expanded state may rely at least predominantly on being supported or held in the expanded state by expanded expandable annular elements of the prosthesis.

The expandable annular elements and the biodegradable tubular element may be attached or connected to each other in any manner or combination of manners. In one embodiment, the expandable annular elements and the biodegradable tubular element are attached to each other by one or more sutures about the circumference of an expandable annular element. In another embodiment, the expandable annular elements and the biodegradable tubular element are attached to each other by an adhesive at one or more points about the circumference of an expandable annular element. In one embodiment, the expandable annular elements and the biodegradable tubular element are attached to each other by one or more hooks provided by and positioned about the circumference of a expandable annular element. For example, a hook grasping mechanism as disclosed in U.S. Publication No. 2003/0009213 may be used. In a further embodiment, the biodegradable tubular element and expandable annular elements may be fixed to each other using an adhesive. The adhesive may be biodegradable or non-biodegradable. In another embodiment, the expandable annular elements are positioned within the lumen of the biodegradable tubular element but are not physically affixed to each other. When such a prosthesis configuration is crimped onto a balloon delivery catheter and expanded at the delivery site, the expandable annular elements press the overlying sections of the tubular element against the vessel wall, thereby maintaining each of the elements in its desired position.

The wall thickness of the expandable annular elements and/or biodegradable tubular elements of an endoprosthesis according to the invention may, for example, be in the range of about 10 microns to 500 microns, such as in the range of about 10 to 200 microns. In one embodiment, the wall thickness is equal to or less than 200 microns, for example, equal to or less than 125 microns. In one embodiment, the wall thickness is in the range of 20 microns to 125 microns. In another embodiment of the invention, the wall thickness is in the range of 20 to 60 microns. In still another embodiment, the wall thickness is in the range of 50 to 100 microns.

A prosthesis according to the invention may be manufactured in any desired length. For example, a prosthesis according to the invention may have a length in the range of 1 to 4 centimeters, such as about 1.8 centimeters. The device may also be manufactured with any suitable unexpanded radius and potential maximum expanded radius, which will be determined by the radius required for catheter delivery and the expanded radius required to bring the wall of the device into contact with the lumen wall of a blood vessel, such as the coronary artery.

For polymeric components of an endoprostheses according to the invention, one or more drugs may be blended with the polymer melt during the formation of an article and/or soak-loaded into the article. Drugs may include, but are not limited to anti-proliferative drugs, immunosuppressant drugs, anti-inflammatory drugs, e.g., steroidal and non-steroidal anti-inflammatory drugs, anti platelet drugs, anti-migratory drugs, anti-thrombotic drugs, drugs that regress plaque, high density lipoprotein (HDL)-mimetics, peptides, polypeptides, hormones, cytokines, agents that promote endothelial cell growth, prohealing drugs and combinations thereof. Drugs according to the invention may, for example, be small molecules, peptides, polypeptides, and radioisotopes.

As used herein the term drug means any sort of agent or compound that has a desired therapeutic and/or prophylactic effect and/or facilitates the role and/or acceptance of the endoprosthesis in the body. For example, drugs may be directed to treating a condition, such as vulnerable plaque and/or may be directed to preventing prosthesis-induced thrombosis, such as heparin. Agents that promote endothelial cell growth and/or reduce inflammation may be used for the treatment of vulnerable plaque. Exemplary agents promoting endothelial cell growth include, for example, vascular endothelial growth factor (VEGF) and estradiols, such as 17-beta-estradiol.

Metallic or non-metallic components of endoprostheses according to the invention may be coated with one or more polymer coatings. The coating(s) may optionally include or be loaded with drugs useful for treating vulnerable and/or for facilitating the desired functioning of the implanted endoprosthesis, for example, anti-thrombotic agents such as heparin to inhibit endoprosthesis-induced thrombosis at the treatment site. U.S. Pat. No. 5,624,411 teaches methods of coating intravascular stents with drugs, and is hereby incorporated by reference in its entirety.

A further embodiment of the invention provides a method for treating vulnerable plaque in a patient in need thereof that includes the step of deploying any of the prostheses described herein at the site of a vulnerable plaque lesion in the patient. Preferably, the prosthesis is positioned so that the tubular element at least partially covers a section of blood vessel that has the vulnerable plaque lesion. The deployment involves an expansion of the radius of the device to that the prosthesis comes into contact with the vessel wall. Contact with the vessel wall provides embolic protection and promotes re-endothelialization, thereby passivating the vulnerable plaque. The tubular element erodes in time and the expandable annular elements, if they are non-biodegradable or not yet fully degraded, become disposed in the surrounding tissue (so that they surround the blood vessel wall) or remain in contact with the vessel wall.

The endoprosthesis may be delivered in a decreased radius configuration on a delivery catheter. The endoprosthesis may be crimped on or otherwise positioned around an inflatable deployment balloon, so that expansion of the balloon at least partially expands the endoprosthesis to its final working radius, for example, in a coronary artery. For self-expanding versions of the endoprosthesis, use of a delivery balloon is optional. A self-expanding prosthesis may, for example, be restrained in a cylindrical cavity covered by a restraining sheath and deployed by retracting the sheath, as known in the art.

Any of the treatment methods of the invention may include a step of locating a vulnerable plaque lesion to be treated by the endoprosthesis in a patient.

According to the invention, determining the location of a vulnerable plaque in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differences characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated herein in its entirety. A method and system for detecting and localizing vulnerable plaques based on the detection of biomarkers is disclosed in U.S. Pat. No. 6,860,851, which is hereby incorporated by reference herein in its entirety. Angiography using a radiopaque and/or fluorescent dye, for example, as known in the art, may be performed before, during and/or after the step of determining the location of the vulnerable plaque, for example, to assist in positioning the prosthesis in a subject artery.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. An expandable endovascular prosthesis, comprising:
at least two expandable annular elements, each having a central axis; and
an at least substantially tubular biodegradable element having an inner surface, an outer surface and a longitudinal central axis,
wherein the central axis of each of the expandable annular elements and of the tubular biodegradable element are at least substantially aligned.

2. The prosthesis of claim 1, wherein
the expandable annular elements are at least substantially sinuate in form.

3. The prosthesis of claim 1, wherein
at least two of the expandable annular elements are disposed within the tubular biodegradable element.

4. The prosthesis of claim 1, wherein
at least two of the expandable annular elements are disposed about the outer surface of the tubular biodegradable element.

5. The prosthesis of claim 1, wherein
the at least two expandable annular elements are affixed to the tubular biodegradable element.

6. The prosthesis of claim 1, wherein
at least one of the expandable annular elements is at least partially radio-opaque.

7. The prosthesis of claim 1, wherein
at least one of the expandable annular elements is at least partially metallic.

8. The prosthesis of claim 1, wherein
the prosthesis is at least partially balloon expandable.

9. The prosthesis of claim 1, wherein
the at least two expandable annular elements are a least partially self-expanding.

10. The prosthesis of claim 1, wherein
of the at least two expandable annular elements, one is disposed at or near one end of the tubular biodegradable element and another is disposed at or near the opposite end of the tubular biodegradable element.

11. The prosthesis of claim 1, wherein
the tubular biodegradable element is at least partially porous.

12. The prosthesis of claim 1, wherein
the tubular biodegradable element has pores having diameters at least predominantly less than 500 microns.

13. The prosthesis of claim 1, wherein
the tubular biodegradable element has pores having diameters at least predominantly in the range of 20 to 200 microns.

14. The prosthesis of claim 1, wherein
the expandable annular elements are at least substantially non-biodegradable.

15. The prosthesis of claim 1, wherein
the expandable annular elements are biodegradable at a slower rate than the tubular biodegradable element.

16. The prosthesis of claim 1, wherein
at least some of the expandable annular elements consists essentially of non-biodegradable metallic material.

17. The method of claim 16, wherein
the tubular biodegradable element is at least substantially polymeric.

18. The prosthesis of claim 1, wherein
neighboring annular elements are not connected to each other by connective elements.

19. The prosthesis of claim 1, wherein
at least two neighboring annular elements are connected by at least one connective element.

20. The prosthesis of claim 1, wherein neighboring annular elements are connected by at least one connective element.

21. A method for treating vulnerable plaque in a patient in need thereof, comprising the steps of:
deploying a prosthesis according to claim 1 at a site of a vulnerable plaque in blood vessel of a patient.

22. The method of 21, further comprising the step of delivering the prosthesis to the site using a delivery catheter.

23. The method of claim 21, further comprising the step of:
prior to deploying the endoprosthesis, locating the site of the vulnerable plaque.

24. The method of claim 21, wherein
at least two of the expandable annular elements of the prosthesis are disposed within the tubular biodegradable element.

25. The method of claim 21, wherein
at least two of the expandable annular elements of the prosthesis are disposed about the outer surface of the tubular biodegradable element.

26. The method of claim 21, wherein
the at least two expandable annular elements of the prosthesis are affixed to the tubular biodegradable element.

27. The method of claim 21, wherein
at least one of the expandable annular elements of the prosthesis is at least partially radio-opaque.

28. The method of claim 21, wherein
at least one of the expandable annular elements of the prosthesis is at least partially metallic.

29. The method of claim 21, wherein
the prosthesis is at least partially balloon expandable.

30. The method of claim 21, wherein
the at least two expandable annular elements of the prosthesis are a least partially self-expanding.

31. The method of claim 21, wherein
of the at least two expandable annular elements of the prosthesis, one is disposed at or near one end of the tubular biodegradable element and another is disposed at or near the opposite end of the tubular biodegradable element.

32. The method of claim 21, wherein
the tubular biodegradable element of the prosthesis is at least partially porous.

33. The method of claim 21, wherein
the tubular biodegradable element of the prosthesis has pores having diameters at least predominantly less than 500 microns.

34. The method of claim 21, wherein
the tubular biodegradable of the prosthesis has pores having diameters at least predominantly in the range of 20 to 200 microns.

35. An expandable endovascular prosthesis, comprising:
at least two expandable non-biodegradable metallic annular elements, each having a central axis; and
an at least substantially tubular biodegradable polymeric element having a wall and a longitudinal central axis, wherein the wall is porous forming pores at least predominantly 500 microns or less in diameter,
wherein the central axis of each of the expandable annular elements and of the tubular biodegradable element are at least substantially aligned.

36. The prosthesis of claim 35, wherein
neighboring annular elements are not connected to each other by connective elements.

37. The prosthesis of claim 35, wherein
at least two neighboring annular elements are connected by at least one connective element.

38. The prosthesis of claim 37, wherein
neighboring annular elements are connected by at least one connective element.
